(19) United States
(12) Patent Application Publication
(10) Pub. No.: US 2025/0261642 A1
(43) Pub. Date: Aug. 21, 2025

(54) POLYMER COACERAVATES AND METHODS OF DISRUPTING AND REMOVING BIOFILMS

(71) Applicants: Abraham Joy, Copley, OH (US); Apoorva Vishwakarma, Silver Spring, MD (US); Amal Narayanan, Princeton, NJ (US)

(72) Inventors: Abraham Joy, Copley, OH (US); Apoorva Vishwakarma, Silver Spring, MD (US); Amal Narayanan, Princeton, NJ (US)

(21) Appl. No.: 18/857,710

(22) PCT Filed: Apr. 20, 2023
(86) PCT No.: PCT/US2023/019244

§ 371 Date: Oct. 17, 2024

Related U.S. Application Data
(60) Provisional application No. 63/332,904, filed on Apr. 20, 2022.
(60) Provisional application No. 63/338,921, filed on May 6, 2022.

(51) Int. Cl. A01N 43/60; A01N 25/02; A01P 1/00; A61L 2/18; A61L 101/44; A61L 101/46

(52) U.S. Cl. CPC A01N 43/60; A01N 25/02; A01P 1/00; A61L 2/18; A61L 2101/44; A61L 2101/46

(57) ABSTRACT

A polyester coacervate capable of disrupting biofilms formed on substrates and methods of treating contaminated substrates to disrupt and remove biofilms using polyester coacervates without the need for antibiotic therapies or mechanical disruption. The polyester coacervate exhibits tunable viscosity, hydrophobic/hydrophilic, and interfacial properties to disrupt and remove a broad range of biofilms formed on a broad range of substrates.

### CROSS-REFERENCE TO RELATED APPLICATIONS

This application claims the benefit of U.S. Provisional Patent Application Ser. No. 63/332,904 filed Apr. 20, 2022, and U.S. Provisional Patent Application Ser. No. 63/338,921 filed on May 6, 2022, which are incorporated herein by reference in their entirety.

### FIELD OF THE INVENTION

One or more embodiments of the invention provides thermoresponsive polyesters that demonstrate disruption and removal of biofilms from substrates.

### BACKGROUND OF THE INVENTION

In water certain polymers show a transition from a soluble to an insoluble phase with increase in temperature. The temperature at which this transition occurs is called as the lower critical solution temperature (LCST) which is determined using cloud point temperature (T_{CP}) measurements. Examples of such polymers include polyesters, polyacrylamides, polyacrylates, polyoxazolines, Pluronics, RNAs and disordered proteins. Copolyesters with monomers that show thermoresponsive behavior or with monomers that do not provides further options to tailor the Top of the polyesters. In addition, we can also incorporate additional properties into such polymer coacervates.

Thermoresponsive polymers undergo a phase transition at their T_{CP}, at which temperature a shift in the hydrophilic-hydrophobic balance occurs. A liquid-liquid phase separation starts to occur resulting in the formation of coacervate droplets. These coacervate droplets are the polymer-rich phase with approximately 40-60% polymer within the coacervates. The coacervates will coalesce over time to form a dense phase (or condensed phase) which settles to the bottom and a supernatant dilute phase. The coacervate is visually cloudy, while the dense phase appears as a clear phase.

Bacteria attach to surfaces and secrete a matrix of extracellular polymeric substances (EPS) around themselves to form bacterial biofilms. The EPS consists of polysaccharides, proteins, nucleic acids and cellular debris. Biofilms are self-organized, structured communities of bacteria within the self-secreted matrix of EPS or biomacromolecules. They can easily contaminate and proliferate on both biotic and abiotic surfaces, even under conditions of extreme chemical and physical stress, making them a major scourge in both healthcare and industrial settings. A recent, pre-COVID era estimate suggests that biofilms have an economic impact in excess of $4 trillion annually, of which $386.8 billion were attributed to the impact of biofilms on medical and human health, and over $3 trillion was spent mitigating or managing the consequences of biofilms in industrial settings. Given the huge social and economic consequences, preventing biofilm formation, and removing existing biofilms has attracted widespread attention.

Current strategies for biofilm removal involve combinatorial antibiotic therapies and mechanical disruption using water sprays and jets, and physical debridement from surface. The current methods of mechanical removal of biofilms do not completely remove them but cause them to slough off and colonize other surfaces, compounding the problem of biofouling and recurring infections. Moreover, it has been shown that the mechanical properties of biofilms can persist upon treatment with antibiotics and biocides, which disable the metabolically active bacteria, but not the non-growing persister cells. These persister cells are protected by the intact biofilm matrix and can reestablish the biofilm. Most ongoing research efforts have been focused on disabling the living, bacterial component of the biofilms like the development of novel antimicrobial agents: small molecules, peptides, and polymers, bacterial signaling inhibitors, bacteriophages, metabolic inhibitors, etc. Since biofilms are a composite of both bacteria and the extracellular matrix of biopolymers, it has also been increasingly recognized that the viscoelasticity and surface-adhesive properties of biofilms play a major role in their persistence and virulence.

Therefore, there is a significant need for methods that can efficiently remove surface-associated bacterial biofilms, whether in a biomedical context such as in an infected wound or a catheter or in engineering applications such as in water treatment plants and fuel tanks.

### SUMMARY OF THE INVENTION

An embodiment of the present invention provides a method for disrupting and removing a biofilm from a contaminated substrate, the method comprising contacting the biofilm and the contaminated substrate with a polyester coacervate composition.

Another embodiment of the present invention provides a method for disrupting and removing a biofilm from a contaminated substrate as in any embodiment above, wherein the polyester coacervate composition comprises an aqueous solution, and a polyester coacervate dispersed within the aqueous solution.

Another embodiment of the present invention provides a method for disrupting and removing a biofilm from a contaminated substrate as in any embodiment above, wherein the biofilm and the contaminated substrate are exposed to water.

Another embodiment of the present invention provides a method for disrupting and removing a biofilm from a contaminated substrate as in any embodiment above, wherein the polyester coacervate composition includes an additive, wherein the additive is an anti-microbial agent.

Another embodiment of the present invention provides a method for disrupting and removing a biofilm from a contaminated substrate as in any embodiment above, wherein the anti-microbial agent is an antibiotic encapsulated within the polyester coacervate.

Another embodiment of the present invention provides a method for disrupting and removing a biofilm from a contaminated substrate as in any embodiment above, wherein 55 percent or greater of a biomass of the biofilm is removed from the contaminated substrate after being contacted with the polyester coacervate solution.

Another embodiment of the present invention provides a method for disrupting and removing a biofilm from a contaminated substrate as in any embodiment above, wherein from 55 percent to 99.9 percent of a biomass of the biofilm is removed from the contaminated substrate after being contacted.

Another embodiment of the present invention provides a method for disrupting and removing a biofilm from a contaminated substrate as in any embodiment above, wherein an area of the contaminated substrate including the biofilm is decreased by from 55 percent to 99.9 percent after being contacted with the polyester coacervate solution.

Another embodiment of the present invention provides a method for disrupting and removing a biofilm from a contaminated substrate as in any embodiment above, wherein the step of contacting includes coating, spreading, injecting, depositing, flowing, or otherwise bringing the polyester coating coacervate composition into contact with the biofilm.

Another embodiment of the present invention provides a method for disrupting and removing a biofilm from a contaminated substrate as in any embodiment above, wherein the biofilm is formed from one of K. pneumoniae, E. coli, S. aureus, and Pseudomonas including P. aeruginosa, P. fluorescens.

Another embodiment of the present invention provides a method for disrupting and removing a biofilm from a contaminated substrate as in any embodiment above, wherein no mechanical cleaning methods are used to disrupt and remove the biofilm.

Another embodiment of the present invention provides a method for disrupting and removing a biofilm from a contaminated substrate as in any embodiment above, wherein the contaminated substrate is not adversely affected after contact with the polyester coacervate.

An embodiment of the present invention provides a composition comprising an aqueous solution, a polyester coacervate dispersed within the aqueous solution, and an additive.

Another embodiment of the present invention provides a composition as in any embodiment above, wherein the polyester coacervate is represented by Formula I, where R1 is selected from moieties that include an N- or N,N-substituted secondary or tertiary amide, R2 and R3 are independently selected from aliphatic groups, unsubstituted aromatic functional groups, and aromatic groups functionalized with one or more hydroxyl groups, n is from about 10 to about 500, E is from about 60 to about 95, M is from 0 to about 40, C is from about 5 to about 40, and where the sum of M+E+C is 100.

Another embodiment of the present invention provides a composition as in any embodiment above, wherein the polyester coacervate includes a unit represented by, one of: TR-PyrPE, TR-DEPE, TR-IPrPE, TR-CPPE, and TR-nPrPE.

Another embodiment of the present invention provides a composition as in any embodiment above, wherein the additive is encapsulated by the polyester coacervate.

Another embodiment of the present invention provides a composition as in any embodiment above, wherein the additive is an anti-microbial agent.

Another embodiment of the present invention provides a composition as in any embodiment above, wherein the additive is ciprofloxacin.

Another embodiment of the present invention provides a composition as in any embodiment above, wherein the polyester coacervate has a density greater than water.

### BRIEF DESCRIPTION OF THE DRAWINGS

FIG. 1 is a schematic representation of a representative chemical structure of HyPPo-0, containing 80/20 ratio of bMoEt/C3-Cou monomers;

FIG. 2 is an image showing the liquid-liquid phase separation of the dilute phase and dense phase of a polymer coacervate, HyPPo-0;

FIG. 3 is a representation of polymer concentration percentage and relative density of the dilute phase and dense phase to water for HyPPo-0;

FIG. 4 is a representation of a testing apparatus for growing biofilms in a variety of conditions and means of introducing a polymer coacervate to the biofilm;

FIG. 5 is a representation for the calculation of interfacial tensions of dense and dilute phases using Young's equation;

FIG. 6 is a representation of a custom-built JKR setup used to measure the pull-of work of adhesion for relating the work required to fracture the biofilm-substrate interface;

FIG. 7 is a representation of biofilm formation performed in channels formed within glass coverslips for testing compositions and methods according to the present invention;

FIG. 8A is a graphical representation of the reduction in biomass and substratum coverage after a biofilm was treated according to an embodiment of the present invention;

FIG. 8B shows 3D renders of confocal stacks showing biofilms of P. aeruginosa before and after exposure to an embodiment of the present invention;

FIG. 9 is a graphical representation of the reduction in biomass and substratum coverage after a variety of biofilms were exposed to an embodiment according to the present invention;

FIG. 10A is a graphical representation of the reduction in biomass and substratum coverage after a biofilm was treated according to an embodiment of the present invention, 20% PVA, and Sylgard™;

FIG. 10B shows 3D renders of confocal stacks showing biofilms before and after exposure to an embodiment of the present invention, 20% PVA, and Sylgard™;

FIG. 11 is a graphical representation of the reduction in biomass and substratum coverage after a biofilm was treated according to an embodiment of the present invention for a variety of substrates upon which the biofilm was grown;

FIG. 12 is a graphical representation of the interfacial tension of liquid-air interface and OTS-liquid interface for water, dilute phase, and dense phase according to an embodiment of the present invention;

FIG. 13 is a graphical representation of the water contact angle of lens substrate and work of adhesion for biofilms submerged under water and under dilute phase according to an embodiment of the present invention;

FIG. 14 is a graphical representation of the reduction in biomass and substratum coverage after a biofilm was subjected to various flow rates of dilute phase and dense phase according to an embodiment of the present invention; and

FIG. 15 is a graphical representation of the reduction in biomass and substratum coverage after a biofilm was subjected to various flow rates of water.

### DETAILED DESCRIPTION OF ILLUSTRATIVE EMBODIMENTS

In a first aspect, the present invention is directed to compositions for disrupting and removing biofilms comprising polyester coacervates, as described below. In one or more embodiments, compositions for disrupting and removing biofilms may further include additives to further promote disruption, removal, and prevention of further biofilm formation.

The polyester coacervate is not particularly limited and one of ordinary skill in the art will be able to select a suitable polyester coacervate without undue experimentation. In various embodiments, a suitable polyester coacervate may include, without limitation, those described in U.S. Pat. Nos. 9,593,201, 10,106,514, 10,336,923, and 11,033,573, all of which are incorporated herein by reference, including their synthesis procedures.

In one or more embodiments a thermoresponsive polyester is provided. The thermoresponsive polyester may comprise a first amide group having a nitrogen atom and a carbonyl group; a second amide group tethered directly or indirectly through an organic group at the carbonyl group of the first amide group; and the nitrogen atom of the first amide group is part of the backbone of the polyester. It has been found that the thermoresponsive polyesters can undergo a thermally induced reversible hydrophobicity change. The thermoresponsive polyesters have a unique combination of properties not typical among other thermoresponsive polymers. Advantageously, the thermoresponsive polyesters may form a coacervate droplet at certain temperatures. The thermoresponsive polyesters are additionally biodegradable.

In one or more embodiments, thermoresponsive polyesters may be prepared from the polymerization of an amide functional diol compound. Those skilled in the art will recognize that a polyester may be prepared from a diol compound with copolymers containing carboxylic acid groups or ester groups using a polycondensation reaction. In one or more embodiments, a thermoresponsive polyester may be prepared using a carbodiimide-mediated polymerization.

The thermoresponsive polyesters may be prepared from an amide functional diol compound and at least one co-monomer. Suitable co-monomers include dicarboxylic acids, hydroxy acids, and diols. Suitable co-monomers are disclosed in WO 2013/130985, which is incorporated herein by reference in its entirety.

The thermoresponsive polyesters may be characterized by the units that derive from the monomers polymerized to prepare the polyester. In these or other embodiments, the derived units are the mer units that result from the monomers that have been reacted to form the polyester. For example, a thermoresponsive polyester may be prepared from an amide functional diol compound and a dicarboxylic acid. In these or other embodiments, the polyester may comprise units derived from an amide functional diol compound and units derived from a dicarboxylic acid. One or more embodiments may include units that derive from other co-monomers.

In one or more embodiments, the amide functional diol compound may comprise a first amide group having a nitrogen atom and a carbonyl group a second amide group tethered directly or indirectly through an organic group at the carbonyl group of the first amide group; and two organic groups terminated with a hydroxyl group attached to the nitrogen atom of the first amide group.

In one or more, the polymers of the present invention may be referred to as polyesters, or coacervates. In one or more embodiments, the polyesters may be represented by Formula I:

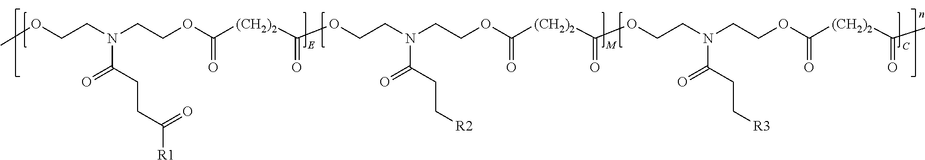

where R1 may be selected from moieties that include an N- or N,N-substituted secondary or tertiary amide, where R2 and R3 may be selected from aliphatic groups, unsubstituted aromatic functional groups, and aromatic groups functionalized with one or more hydroxyl groups, n is from about 10 to about 500, E is from about 60 to about 95, M is from 0 to about 40, C is from about 5 to about 40, and where the sum of M+E+C is 100. In one or more embodiments R1 is selected such that the thermoresponsive polyester includes the following or substantially similar structures:

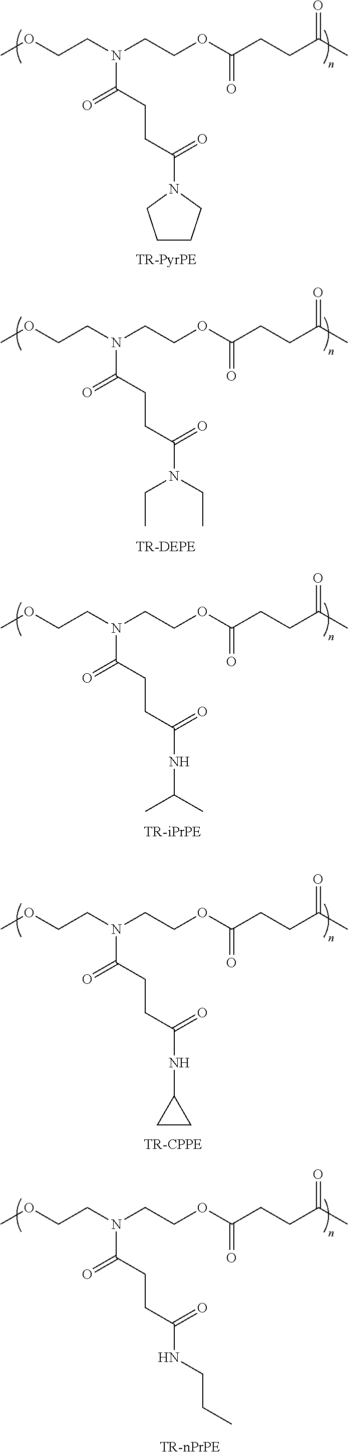

In one or more embodiments, R1 may be selected from 2-ethoxyethan-1-amine, Morpholine, bis(2-methoxyethyl)amine, and 2-methoxyethan-1-amine groups. In one or more embodiments, R2 and R3 may be selected from methyl, benzene, phenol, catechol, resorcinol and pyrogallol groups. In one or more embodiments, the polymers have an LCST in aqueous medium of from about-20 to about 100° C.

Without wishing to be bound by theory, it is believed that R1 exhibits the most impact over the coacervating behavior of a thermoresponsive polyester according to embodiments of the present invention. Furthermore, it is believed that R2 and R3 influence the hydrophobic/hydrophilic balance of the thermoresponsive polyester, as well as the interfacial properties of the thermoresponsive polyester.

In one or more embodiments, the polymers of the present invention may be referred to as ‘Hybrid Protein-like Polyester’ (HyPPo) polymers, or coacervates. In one or more embodiments, the HyPPo polymers may be represented by Formula II:

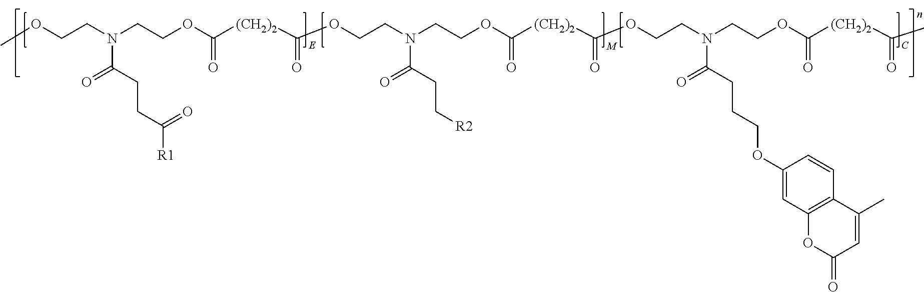

where R1 is selected from moieties that include an N- or N,N-substituted secondary or tertiary amide, R2 is selected from aliphatic groups, unsubstituted aromatic functional groups, and aromatic groups functionalized with one or more hydroxyl groups, n is from about 10 to about 500, E is from about 60 to about 95, M is from 0 to about 40, C is from about 5 to about 40, and where the sum of M+E+C is 100. In one or more embodiments, R1 is selected from 2-ethoxyethan-1-amine, Morpholine, bis(2-methoxyethyl)amine, and 2-methoxyethan-1-amine groups. In one or more embodiments, R2 is selected from methyl, benzene, phenol, catechol, resorcinol and pyrogallol groups. In one or more embodiments, the HyPPo polymers have an LCST in aqueous medium of from about-20 to about 100° C.

In one or more embodiments, the HyPPo polymers include a tropoelastin-mimetic domain (E), a mussel-inspired (M) domain that includes a catechol functional group, and a cross-linking (C) domain. Examples of HyPPo polymers may be represented by Formula III:

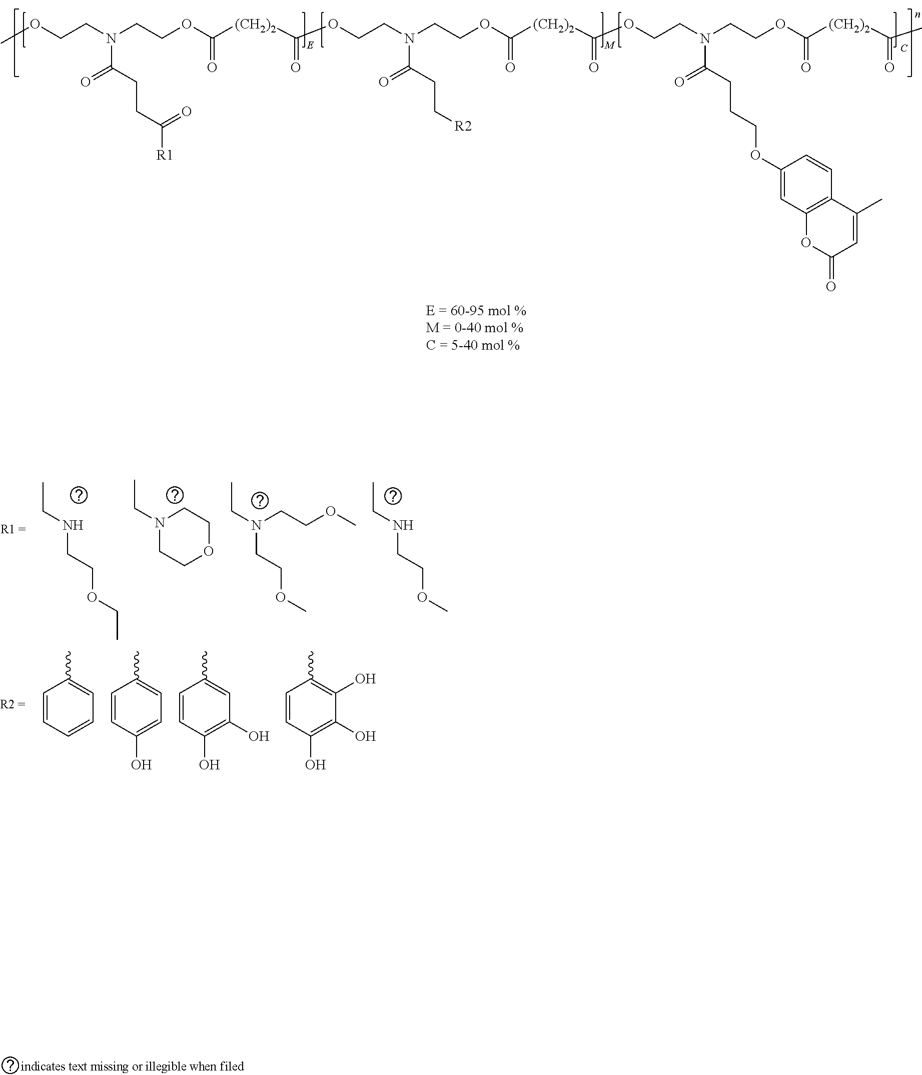

In one or more embodiments, HyPPo polymers comprise a class of statistical copolyesters that may be prepared from two or more N-functionalized diols, such as diethanolamides, and one or more diacid, such as succinic acid, and may be synthesized via polyesterification reactions. An exemplary reaction scheme is shown in Scheme 1.

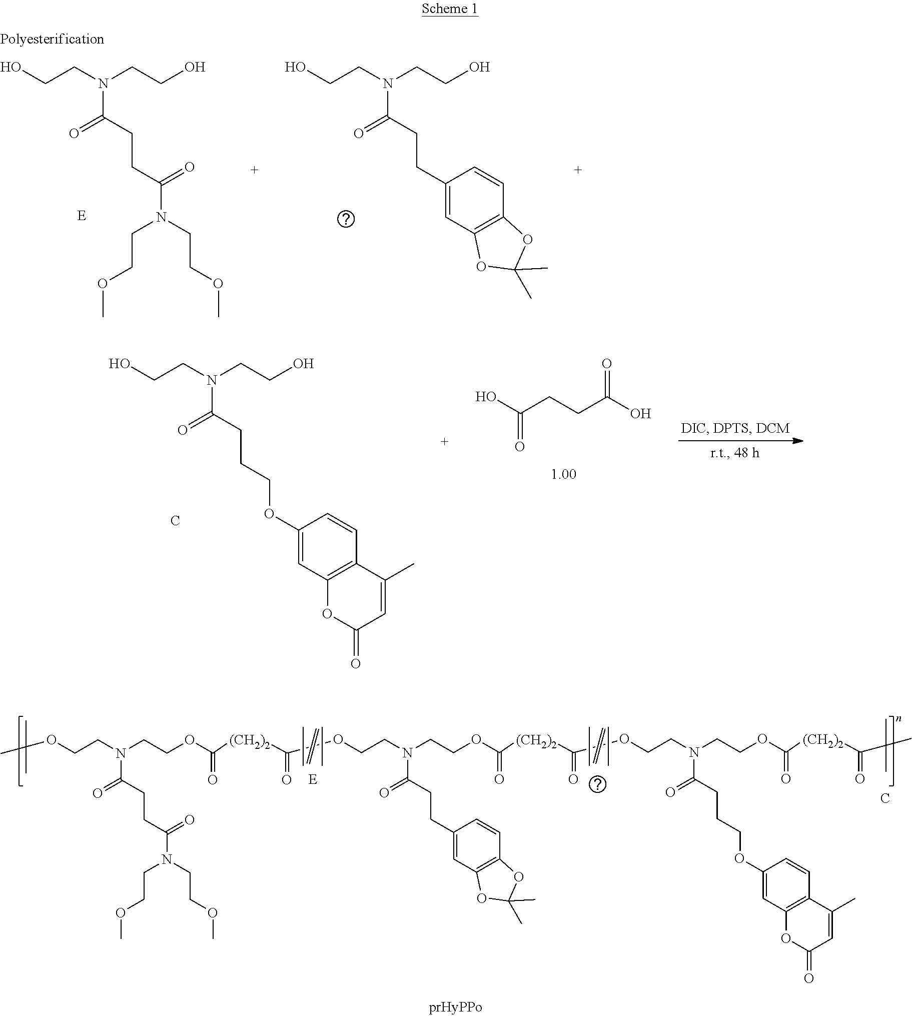

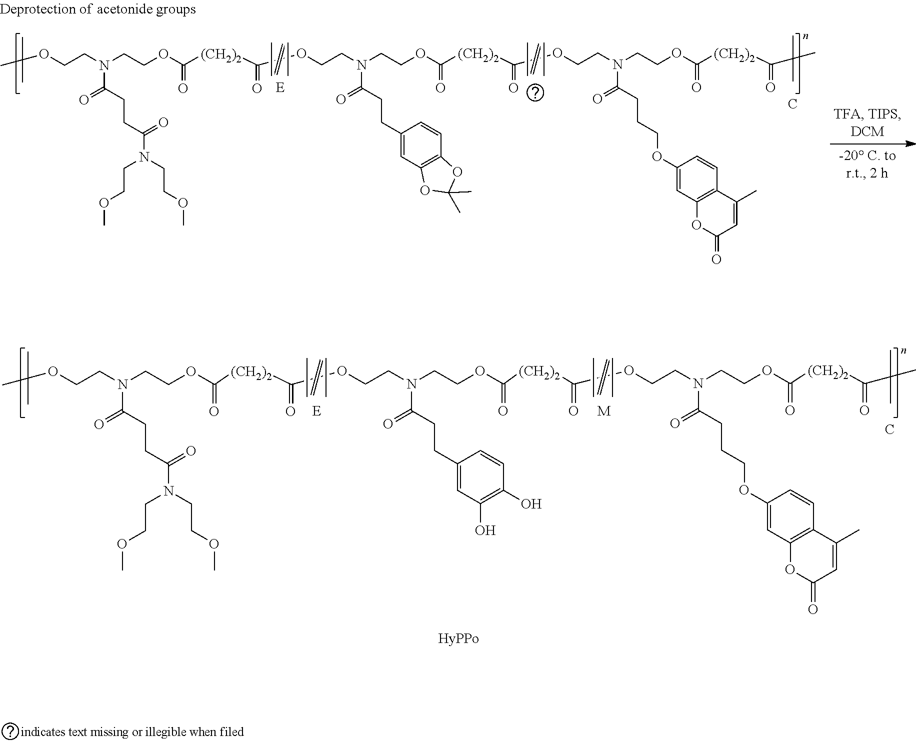

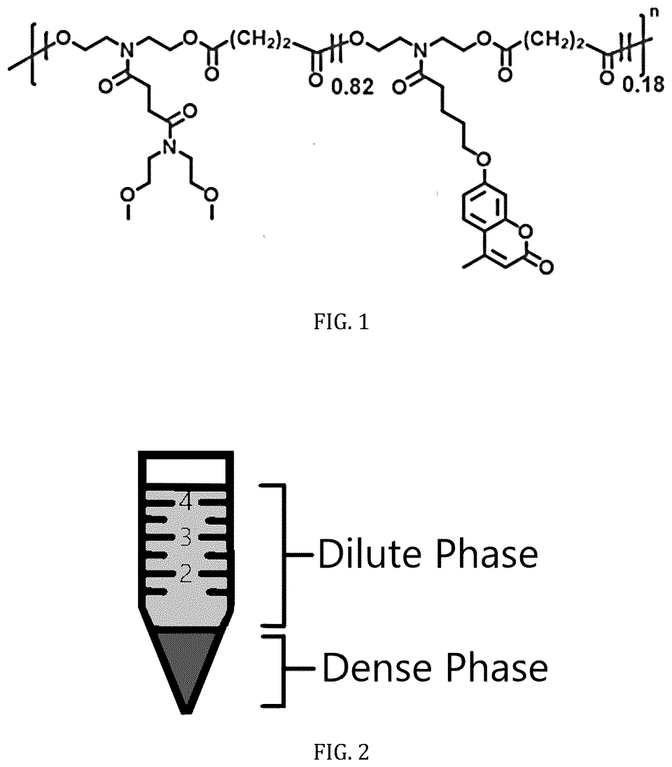

In some embodiments, polyester coacervates according to the present invention may not contain any catechol (DOPA) pendant groups. In such embodiments, the polyester may show comparable adhesion strength to the other catechol-containing polyester coacervates. For example, HyPPo-0, containing 80/20 ratio of bMoEt/C3-Cou monomers, as shown in FIG. 1, which does not contain any catechol (DOPA) pendant groups, demonstrates comparable adhesion strength to the other catechol containing polyester coacervates.

Without wishing to be bound by theory, it is believed selection of a suitable polyester coacervate is dependent upon the type of biofilm and associated bacteria, and the type of substrate upon which the biofilm is formed. As discussed above, biofilms are formed from bacterial secretions of bacteria attached to a substrate and may comprise a matrix of EPS formed around the bacteria. The EPS may include polysaccharides, proteins, nucleic acids, and cellular debris. Biofilms provide for the self-organization and structuring of bacterial communities within the self-secreted matrix of EPS or biomacromolecules. Thus, the composition of the biofilm may play a significant role in the viscoelasticity and surface-adhesive properties of the biofilm. Further, the type of substrate upon which the biofilm forms influences the surface-adhesive properties of the formed biofilm. Differences in substrate surface energies may result from, without limitation, the surface including different functionalities including hydrophobicity and hydrophilicity. Accordingly, suitable polyester coacervates may be selected to target these biomechanical and interfacial properties of a target biofilm formed on a substrate.

Significant removal of biomass in mature biofilms has been accomplished using mechanical shear. Biofilms grown under fluid flow and high shear stresses are generally known to be thinner, denser, and more resistant to external mechanical perturbations. Accordingly, a variety of factors contribute to whether a polyester is suitable for use in the compositions according to the present invention.

Without wishing to be bound by theory, it is believed that suitable polymers may form an appropriate structure that has the ability to form an intimate contact with the biofilm and/or substrate surface yet move across the biofilm and/or substrate surface. For this to be possible, the polyester structure has to have an appreciable amount of water contained in these structures that provide a balance between surface contact and surface rolling. Further, it is believed that suitable polymers may form interactions with the biofilm constituents such as polymer-polysaccharide or polymer-protein interactions, this would assist in the displacement of the biofilm. Therefore, the combination polymer-biofilm interactions combined with strong polymer-surface interactions will assist in the removal of the biofilm.

In one or more embodiments, suitable thermoresponsive polyesters may include a thermoresponsive monomer and an additional monomer. In some embodiments, the additional monomer may be hydrophilic. In other embodiments, the additional monomer may be hydrophobic.

In one or more embodiments, the thermoresponsive polyester may be characterized by an advantageous lower critical solution temperature (LCST), which may be measured using dynamic light scattering to determine the cloud point temperature for thermoresponsive polyester that form coacervates. Advantageously the LCST of the thermoresponsive polyester may be tuned by selecting different amide groups and by the selection of copolymers. In one or more embodiments, the thermoresponsive polyester is characterized by a LCST that is at least 5° C., in other embodiments at least 7° C., and in other embodiments at least 45° C. In these or other embodiments, the thermoresponsive polyester is characterized by a LCST that is at most 50° C., in other embodiments at most 90° C., and in other embodiments at most 95° C. In certain embodiments the thermoresponsive polyester may be characterized by a LCST that is from about 5° C. to about 95° C., in other embodiments from about 7° C. to about 90° C., and in other embodiments from about 45° C. to about 50° C.

In one or more embodiments, a thermoresponsive polyester may form a coacervate in an aqueous solution. In one or more embodiments, an aqueous solution is a solution where the solvent is water. In other embodiments, an aqueous solution is a solution where the predominant solvent is water. In other embodiments, an aqueous solution is a solution where there is a sufficient amount of water present to allow the thermoresponsive polyester to form a coacervate. Aqueous solutions may also include, but are not limited to, biological systems, buffered systems such as phosphate buffered saline, saline, and serum.

Generally, coacervates may form at a temperature above the LCST. While not wishing to be bound by any particular theory, it is believed that coacervates are formed when there is an incomplete dehydration of the water molecules surrounding the thermoresponsive polyester at a temperature above the LCST. Once formed, coacervates may coalesce into precipitated polyester phases when they are centrifuged or when allowed to settle. Coacervates are advantageous, because they may be used to encapsulate additives suitable for a variety of purposes.

Molecules suitable for encapsulation as additives include small molecules, oligomers, and macromolecules. Suitable small molecules for encapsulation as additives include active pharmaceutical ingredients, fluorescent dyes, and imaging agents. Suitable oligomers for encapsulation as additives include peptides, oligonucleotides, and oligosaccharides. Suitable macromolecules for encapsulation as additives include polysaccharides, and synthetic polymers.

In some embodiments, additives may be encapsulated in the polyester coacervate composition during formation of the polyester coacervate. In some embodiments, this includes incorporating the additive into the dense phase by dissolving the additive along with the polyester while below T_{CP} and then raising the temperature above T_{CP}.

Such polyester coacervates can also be incorporated with antibacterial and antimicrobial therapeutics to remove biofilms and kill bacteria in the process. The above polyester dense phase is not designed to be antibacterial; it only disrupts and removes bacterial biofilms. In this process the antibiotic will be incorporated within the coacervates and subsequently in the dense phase upon coalescence of the coacervates. Use of the antibiotic encapsulated dense phase will kill the bacteria that have been displaced from the biofilm by the coacervate as the antibiotic will have better access to the bacteria when they are not encased within the biofilm.

In one or more embodiments, a thermoresponsive polyester may be used to purify an molecule in solution by mixing a thermoresponsive polyester into a solution containing a molecule to be purified, raising the temperature of the solution above the LCST of the thermoresponsive polymer to form a coacervate and encapsulate the molecule to be purified, and precipitating the coacervate. The coacervate may precipitate, for example, by centrifugal force. The solution may then be decanted from the precipitate and the coacervate may be cooled to release the molecule. In one or more embodiments, the thermoresponsive polyester may include a functional group with a suitable molecular specificity. In these or other embodiments, a functional group with molecular specificity may be imparted into the polymer by preparing a thermoresponsive polyester with a functional diol or a functional dicarboxylic acid. For example, in these or other embodiments, a thermoresponsive polyester may include a charged functional group to be selective for charged proteins. In other embodiments, a thermoresponsive polyester may include a hydrophilic functional group to be selective for water soluble proteins.

According to the present invention, the polyester dense phase or condensates included in compositions according to the present invention may be utilized as a tool for the removal of biofilms from substrates. Without wishing to be bound by theory, the removal is believed to be facilitated by the physical and chemical properties of the polyester dense phase. The low interfacial energy of the dense phase enables it to be spread onto a surface that is hydrated or submerged in water. Although the condensate includes a large amount of water, the low interfacial energy allows it to form intimate contact with an underwater surface. Condensates can spread on a surface submerged underwater and form strong adhesive bonds with the surface. According to some embodiments of the present invention, the property allowing coacervates to form intimate contact and spread over an underwater surface also allows it to disrupt and remove bacterial biofilms from a surface or substrate.

In another aspect, the present invention is directed to methods of disrupting and removing biofilms from a contaminated substrate, as described below. In one or more embodiments methods for disrupting and removing a biofilm from a contaminated substrate includes contacting the biofilm with a composition including a polyester coacervate as described above.

In some embodiments, the composition including a polyester coacervate may include an aqueous solution and a polyester coacervate dispersed within the aqueous solution. In some embodiments, the composition may further include an additive. In some embodiments, the additive may be encapsulated within a polyester coacervate. In some embodiments, the additive may include an anti-microbial agent. In some embodiments, the additive may be an antibiotic.

In some embodiments, the contaminated substrate includes a glass surface. In some embodiments, the contaminated substrate includes a functionalized surface. In some embodiments, the contaminated substrate includes a hydrophobic surface. In some embodiments, the contaminated substrate includes a hydrophilic surface. In some embodiments, the contaminated substrate may be under water.

The efficacy of disruption and removal of biofilms according to methods of the present invention may be characterized according to the biomass of a biofilm removed from a contaminated substrate. In some embodiments, the amount of biomass of the biofilm removed after being contacted with the polyester coacervate solution is 55 percent or greater. In some embodiments, 60 percent or greater. In some embodiments, 65 percent or greater. In some embodiments, 70 percent or greater. In some embodiments, 75 percent or greater. In some embodiments, 80 percent or greater. In some embodiments, 85 percent or greater. In some embodiments, 90 percent or greater. In some embodiments, 95 percent or greater. In some embodiments, 96 percent or greater. In some embodiments, 97 percent or greater. In some embodiments, 98 percent or greater. In some embodiments, 99 percent or greater. In some embodiments, 99.9 percent or greater.

In some embodiments, the amount of biomass of the biofilm removed after being contacted with the polyester coacervate solution is from 55 percent to 99.9 percent. In some embodiments, from 60 percent to 99.9 percent. In some embodiments, from 65 percent to 99.9 percent. In some embodiments, from 60 percent to 99.9 percent. In some embodiments, from 70 percent to 99.9 percent. In some embodiments, from 75 percent to 99.9 percent. In some embodiments, from 80 percent to 99.9 percent. In some embodiments, from 85 percent to 99.9 percent. In some embodiments, from 90 percent to 99.9 percent. In some embodiments, from 95 percent to 99.9 percent.

The efficacy of disruption and removal of biofilms according to methods of the present invention may be characterized according to the area of the contaminated substrate including the biofilm that is decreased after being contacted with a polyester coacervate. In some embodiments the area of the contaminated substrate including the biofilm that is decreased after being contacted with a polyester coacervate is from 55 percent or greater. In some embodiments, 60 percent or greater. In some embodiments, 65 percent or greater. In some embodiments, 70 percent or greater. In some embodiments, 75 percent or greater. In some embodiments, 80 percent or greater. In some embodiments, 85 percent or greater. In some embodiments, 90 percent or greater. In some embodiments, 95 percent or greater. In some embodiments, 96 percent or greater. In some embodiments, 97 percent or greater. In some embodiments, 98 percent or greater. In some embodiments, 99 percent or greater. In some embodiments, 99.9 percent or greater.

In some embodiments the area of the contaminated substrate including the biofilm that is decreased after being contacted with a polyester coacervate is from 55 percent to 99.9 percent. In some embodiments, from 60 percent to 99.9 percent. In some embodiments, from 65 percent to 99.9 percent. In some embodiments, from 60 percent to 99.9 percent. In some embodiments, from 70 percent to 99.9 percent. In some embodiments, from 75 percent to 99.9 percent. In some embodiments, from 80 percent to 99.9 percent. In some embodiments, from 85 percent to 99.9 percent. In some embodiments, from 90 percent to 99.9 percent. In some embodiments, from 95 percent to 99.9 percent.

Akin to the cleaning strategies employed by barnacles, polyester coacervates possess unique properties, viz. optimal viscosity, density greater than water, and low surface energy; that enable them to sink to the bottom of a surface underwater, rapidly spread on the surface and displace existing biofilms from the surface. Both the dense and dilute phase reduce the work of adhesion required to fracture the biofilm-substrate interface.

In light of the foregoing, it should be appreciated that the present invention significantly advances the art by providing polyester coacervates for disrupting and removing biofilms that is structurally and functionally improved in a number of ways. While particular embodiments of the invention have been disclosed in detail herein, it should be appreciated that the invention is not limited thereto or thereby inasmuch as variations on the invention herein will be readily appreciated by those of ordinary skill in the art. The scope of the invention shall be appreciated from the claims that follow.

### EXAMPLES

In order to demonstrate the practice of the present invention, the following examples have been prepared and tested. The examples should not, however, be viewed as limiting the scope of the invention. The claims will serve to define the invention.

Materials and reagents used in the following examples are described below. All materials and solvents were used as received unless otherwise noted. Sodium phosphate dibasic, potassium chloride, potassium phosphate monobasic, iron (II) sulfate, ammonium sulfate, magnesium sulfate, polyvinyl alcohol (PVA, 98-99% hydrolyzed, Mw 31 k to 50 k) and agar powder were purchased from Sigma-Aldrich (St Louis, MO, USA). Diethanolamine, rhodamine B, and D(+)-glucose were obtained from Alfa Aesar (Ward Hill, MA, USA). Sodium chloride was purchased from BDH Chemicals (Dubai, UAE). Anhydrous methanol, ethanol, and dimethylformamide (DMF) were purchased from EMD Millipore (Billerica, MA, USA). N,N′-Diisopropylcarbodiimide (DIC) was purchased from Chem-Impex International (Wood Dale, IL). Methylene chloride (DCM) was first dried with CaH₂, after which, it was distilled and stored over molecular sieves. CDCl₃ was purchased from Cambridge Isotope Lab. Trypticase Soy Broth (TSB) and syringes (1 mL, 5 mL, and 50 mL) were purchased from Becton-Dickinson (Franklin Lakes, NJ, USA). Casamino acids, lysogeny broth (LB), and succinic acid were ordered from Fisher Scientific (Hampton, NH, USA). 4-(Dimethylamino)pyridinium-4-toluene sulfonate (DPTS) was synthesized based on a previously reported protocol. SYLGARD™ 184 Part A was purchased from Dow Inc. (Midland, MI, USA). Phenethyltrichlorosilane (PETS), (2-[methoxy (polyethyleneoxy)₉₋₁₂propyl]trimethoxysilane (PEG) and n-octadecyltrichlorosilane were purchased from Gelest Inc (Morrisville, PA, USA). VWR VistaVision™ Cover Glasses (No. 1.5, L×W=24×50 mm) were purchased from VWR International (Radnor, PA). Thermo Scientific™ Nalgene™ Pharma-Grade Platinum-Cured Silicone Tubing (ID=1.6 mm, OD=3.2 mm) was used for flow experiments. The connectors (Y, straight, 4-way; ID=1.6 mm) were purchased from US Plastics Corp. (Lima OH). DMAO for staining bacteria was purchased from Biotium (Fremont, CA, USA). Escherichia coli (ATCC 25922), Pseudomonas aeruginosa PAO1, and Staphylococcus aureus (ATCC 25923) were purchased from American Type Culture Collection (ATCC). Klebsiella pneumoniae was kindly donated by Dr. Hazel A Barton (University of Akron, OH, USA).

Characterizations of the following examples were prepared using the following techniques and instrumentation. A Varian Mercury 300 MHz spectrometer was used to acquire the NMR spectra. The 1H NMR chemical shifts were reported in ppm relative to the residual protons signal in the deuterated solvent. Polymer molecular weight was measured via size exclusion chromatography (SEC) on a TOSOH EcoSec HLC-8320 instrument equipped with an RI detector and two PSS Gram Analytical SEC columns in series using 25 mM LiBr in N,N-dimethylformamide (DMF) as the mobile phase (flow rate=0.8 mL/min). The column and detector were maintained at 50° C. and the molecular weights were estimated using a polystyrene standard curve. Labconco FreeZone Plus 2.5 L Lyophilizer was used to freeze-dry dense and dilute phases. The cloud point temperature (T_{CP}) of the polymer was measured using a Shimadzu UV-1800 UV-Vis spectrophotometer equipped with a Shimadzu S-1700 thermoelectric single-cell holder in a 1.0 cm quartz cell with a nitrogen chamber. TA Instruments ARES-G2 rheometer was used to perform viscosity measurements. Olympus FLUOVIEW FV1000 laser scanning microscope (LSM) with FLUOVIEW software package was used to image the biofilm samples and obtain 3D renders of biofilm Z-stacks. Rame′-hart contact angle goniometer equipped with drop image software package was used for surface tension experiments. A Hach DR 2800™ Spectrophotometer was used to determine the optical density (OD600) of bacterial cultures. NE-300 Just Infusion™ Syringe Pumps (Syringepump.com) were used for flow experiments.

A polymer coacervate referred to as HyPPo-0, containing 80/20 ratio of bMoEt/C3-Cou monomers, as shown in FIG. 1, was synthesized according to the procedures described below. In short, N¹,N¹-bis(2-hydroxyethyl)-N⁴,N⁴-bis(2-methoxyethyl) succinimide or bMoEt (6.547 g, 20.43 mmol, 0.8 eq.), N,N-bis(2-hydroxyethyl)-4-((4-methyl-2-oxo-2H-chromen-7-yl)oxy) butanamide or C₃-Cou (1.783 g, 5.10 mmol, 0.2 eq.), succinic acid (3.6196 g, 30.65 mmol, 1.2 eq.), and DPTS (3.0075 g, 10.22 mmol, 0.4 eq.) were added to a 100 mL round bottom flask. For tagging the polymer with rhodamine, rhodamine B (0.0004 g, 0.0008 mmol) was added to the reaction mixture as well. The round bottom flask contents were kept under vacuum for 15 min and backfilled with N₂ at least three times to remove moisture and volatile impurities. The reactants were dissolved in anhydrous DCM (50 mL), and then DIC (15.9 mL, 102.17 mmol, 4 eq.) was added dropwise to the reaction mixture maintained at 0° C. The reaction was stirred at room temperature for 48 h, after which the reaction mixture was filtered and dried under reduced pressure. This mixture was then dissolved in methanol (˜20 mL) and dialyzed against methanol using a regenerated cellulose membrane (MWCO=3.5 kDa) for 72 h. The dialyzed polymer solution was later dried under reduced pressure to yield a white (magenta, in the case of rhodamine-tagged) solid polymer. The polymer was characterized using 1H NMR and GPC (Mn=14.8 kg/mol, PDI=2.65, Mw=39.1 kg/mol).

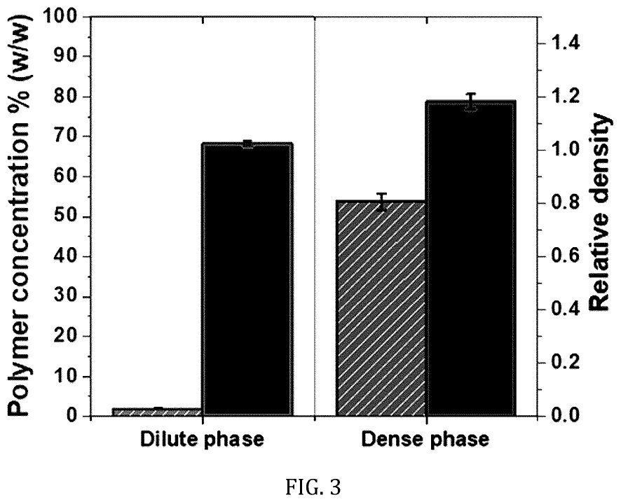

The dense and dilute phases, from the aqueous polymer solution were obtained as follows. The polymer (1 g, T_{CP}=10.8° C.) was dissolved in cold water (10 mL, T_{water}˜4° C.) by vigorous vortexing in a 15 mL centrifuge tube. Once the solution was homogeneous, it was allowed to warm up to room temperature (T˜20° C.) overnight, where it initially turned cloudy, and over time separated into two distinct phases: dilute phase (top) and dense phase (bottom), as shown in FIG. 2. Both the dilute and dense phases can be easily extracted using a micropipette. The polymer concentration, in percent weight of polymer per weight of solution and relative density for both dilute phase and dense phase are shown in FIG. 3.

The T_{CP} of the polymer was measured according to the following. An aqueous polymer solution (10 mg/mL) was prepared and stored overnight at 4° C. The T_{CP} measurements were performed on a Shimadzu UV-1800 UV-vis spectrophotometer with a Shimadzu S-1700 thermoelectric single-cell holder in a 1 cm quartz cell. 18.2 MΩ/cm ultrapure water was used as a reference. The polymer solution was transferred to the pre-cooled quartz cuvette, which was then kept at 3° C. for 3 min under N₂ flow for thermal equilibration, after which the absorbance was measured at λ=500 nm at a temperature ramp rate of 1° C./min until 30° C. Each experiment was performed in triplicate. T_{CP} was designated as the temperature at which the normalized absorbance reached 50%.

Rectangle (24×50 mm, #1.5) and round glass (diameter, d=18 mm, #1.5) coverslips were solvent-cleaned using sonication with toluene, acetone, and ethanol, followed by piranha treatment. The coverslips were then washed with ultrapure water (volume resistivity=18.2 MΩ·cm) and blow-dried using a stream of N₂. After drying, the coverslips were treated with air-plasma to remove residual contaminants and immediately transferred into a sealed flask containing 0.1% v/v silanes (phenethyltrichlorosilane, 2-[methoxy (polyethyleneoxy)₉₋₁₂ propyl]trimethoxysilane or n-octadecyltrichlorosilane) in anhydrous toluene with continuous N₂ bubbling. After the overnight reaction, the coverslips were rinsed thoroughly with toluene, acetone, and ethanol, and then dried in a stream of N₂, and annealed overnight at 120° C. under reduced pressure before use. Round glass coverslips (d=18 mm, #1.5) were solvent cleaned and then cut in the middle to create a 3 mm gap. Each half of the coverslip was glued onto another round glass coverslip (d=18 mm, #1.5) to create a 3 mm channel in the middle. These channel coverslips were exposed to ozone plasma to remove surface impurities and then used immediately for microbiological experiments.

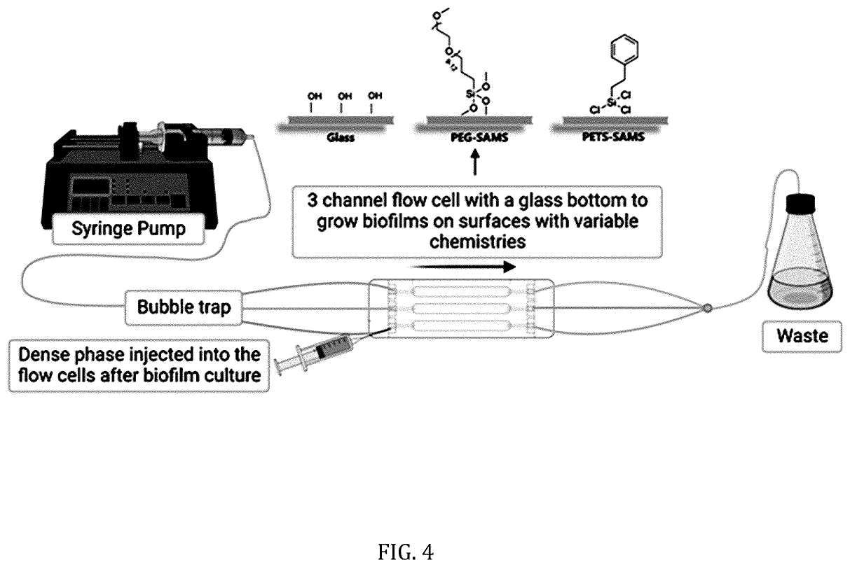

The 3-channel biofilm flow cell, as shown in FIG. 4, was based on a design with minor modifications. Each channel measures 40 mm×4 mm×4 mm. Glass coverslips (L×W=24×50 mm, #1.5) were solvent cleaned followed by piranha treatment and ozone plasma and/or reacted with silanes to create SAMs as mentioned above. The glass coverslips were then glued on top of the flow cell using a commercial silicone sealant. The flow cells were sterilized by rinsing with 70% ethanol and sterile water inside a biosafety cabinet. The rest of the components of the flow setup were either purchased sterile or sterilized by autoclaving before assembly.

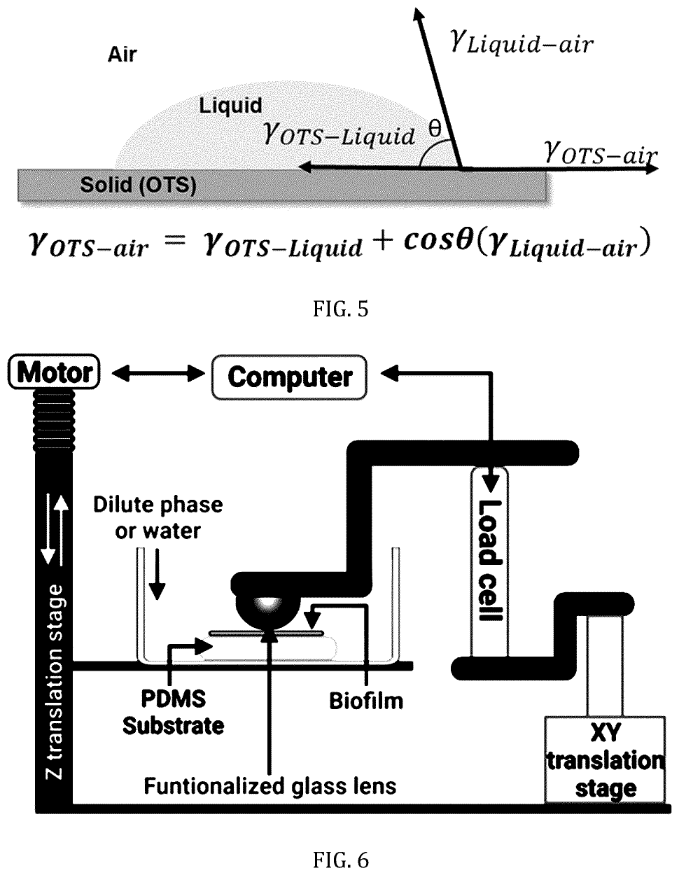

Three (3) mL BM2 Minimal Media [BM2 MM; 62 mM pH 7 potassium phosphate buffer, 0.5 mM MgSO₄, 10 μM FeSO₄, 0.4% (w/v) glucose, 0.5% (w/v) casamino acids], was inoculated with an overnight culture of bacteria in LB and incubated at 37° C. with shaking (200 rpm) for 3-4 h until the culture reached the mid-log phase. This bacterial suspension was diluted to ˜0.1 OD₆₀₀ and further diluted to obtain a working concentration of ˜10⁶ CFU/mL in BM2 MM, 3 mL of which was used to inoculate each well of a 6-well plate containing channel coverslips constructed as described above. The well plate was incubated at 37° C. for 24 h under stationary conditions to grow biofilms. The next day, each coverslip was carefully transferred to a 35 mm petri dish containing 3 mL of sterile physiological saline (PS, 0.9% aq. NaCl), and washed again with 3 mL PS using a micropipette to remove unattached bacteria. The biofilm was then stained with DMAO for 15 min in the dark as per the manufacturer's instructions, after which the coverslips were washed again with PS to remove excess DMAO. Then, 20 μL of dense phase was pipetted on one end of the channel coverslip, while immersed in PS. The petri dish containing the channel coverslips was gently put into a 10° incline to allow the dense phase to ‘roll off’ the biofilm on the channel as shown in FIG. 5-2A. The biofilm on the 3 mm channels on the coverslips were then imaged using a confocal microscope with a 20× objective, and a 488 nm laser line was used to excite DMAO. The acquired images were analyzed using the COMSTAT plugin in ImageJ to quantify biomass and surface coverage.

The flow setup was assembled as shown in FIG. 4 and attached to a syringe pump. The assembled flow setup was primed by pumping BM2 MM at 9 mL/h for 2 h, after which the flow was stopped, and the inlets and outlets of the flow cells were clamped. 400 μL of ˜0.02 OD₆₀₀ dilution of a mid-log culture of P. aeruginosa PAO1 in BM2 MM was used to inoculate each channel of the flow cell using a syringe. The flow cells were kept facing the coverslip side down, and the bacteria were allowed to adhere to the glass surface for 2 h at 37° C. After 2 h, the flow cell inlets and outlets were unclamped, and the media was pumped through the setup at 3 mL/h for 70 h at 37° C. to allow the biofilms to grow under hydrodynamic conditions. After 70 h, the BM2 MM flow was stopped and sterile PS was pumped through the setup at 6 mL/h for 1.5 h, after which the biofilms in the flow cells were stained by injecting 400 μL of DMAO solution into each channel and incubating for 15 min in the dark. Excess DMAO was removed by flushing the channel with 500 μL sterile PS and then each channel was imaged at 3 random spots using a confocal microscope. Once the ‘before’ images were acquired for each channel. The flow cells were placed on a flat surface with the glass coverslip face down and 50 μL of dense phase or 20% PVA or Sylgard™ were injected from the inlet into each channel. The flow cell was left undisturbed for 10 min, after which it was gently placed on a 10° incline and monitored until the dense phase moved to the outlet (˜10 min). Then, the dense phase or controls were removed from the channel by slowly flowing 500 μL sterile PS and each channel was imaged again using a confocal microscope at three random positions. For the dilute phase, the flow cell was connected to a 12 mL syringe filled with the dilute phase and the dilute phase was pumped into each channel at the desired flow rate for a time (adjusted to ensure the volume of the dilute phase pumped remained constant), after which the channels were visualized under a confocal microscope immediately.

The contact angle measurements were performed on a ramé-hart contact angle goniometer equipped with DROPimage software. Briefly, 8 μL of liquid was brought in contact with the surface using a micropipette, and the left and right water contact angles at the three-phase contact line were measured every second for the first 30 s. The reported contact angles were mean±standard deviation of both left and right contact angles of at least three replicates. The surface tension of water, dense and dilute phase were measured using pendant drop shape fitting, and the interfacial tensions of dense and dilute phases on OTS were calculated by using Young's equation as shown in FIG. 5.

A custom-built JKR setup was used to measure the pull-off work of adhesion which is analogous to the work required to fracture the biofilm-substrate interface as shown in FIG. 6. Three (3) mL of ˜10⁶ CFU/mL P. aeruginosa PAO1 in BM2 MM was used to inoculate each well of a 6-well plate containing sterile PDMS sheet (L×W×H=10 mm×10 mm×3 mm, elastic modulus, G′=0.7 MPa). The well plate was incubated at 37° C. for 5 h in a stationary incubator to allow biofilm growth. To validate the presence of biofilm on the PDMS sheet, DMAO was used to stain bacteria as discussed above and visualized under a microscope. Then, the PDMS sheet containing biofilm was carefully washed with water to remove unattached bacteria and transferred to a 30 mm glass petri dish (depth=20 mm) containing ˜20 mL (18.2 MΩ/cm ultrapure water or dilute phase), such that the biofilm on top was completely submerged in liquid. Hemispherical glass lenses (radius of curvature 1.25 mm) along with silicon wafers were functionalized with OTS as mentioned above. To create a lens surface with variable contact angles, the OTS-functionalized lenses and silicon wafers were exposed to ozone plasma for a specific time and the corresponding silicon wafers were used to measure the water contact angle. The hemispherical lenses were then attached to an L-arm with 1 N load cell and connected to an XY translating stage. The lens was slowly lowered (preload=1 mM, loading speed=200 nm/s) to bring in contact with the biofilm underwater, equilibrated for 60 s, and then the pull-off forces were measured (unloading speed=40 nm/s). Experiments were performed in triplicates and each data point was reported as mean±standard deviation.

The viscosity of dense phase and viscosity controls were measured on a TA ARES-G2 rheometer². The dense phase (or comparative samples of 20% aq. PVA or Sylgard™) was loaded onto a custom-built aluminum cup (d=30 mm, h=5 mm) serving as the bottom plate and a 0.04 rad aluminum cone (d=30 mm) was used as the top plate. The viscosity was plotted as a function of the shear rate.

### Example 1—P. aeruginosa Study

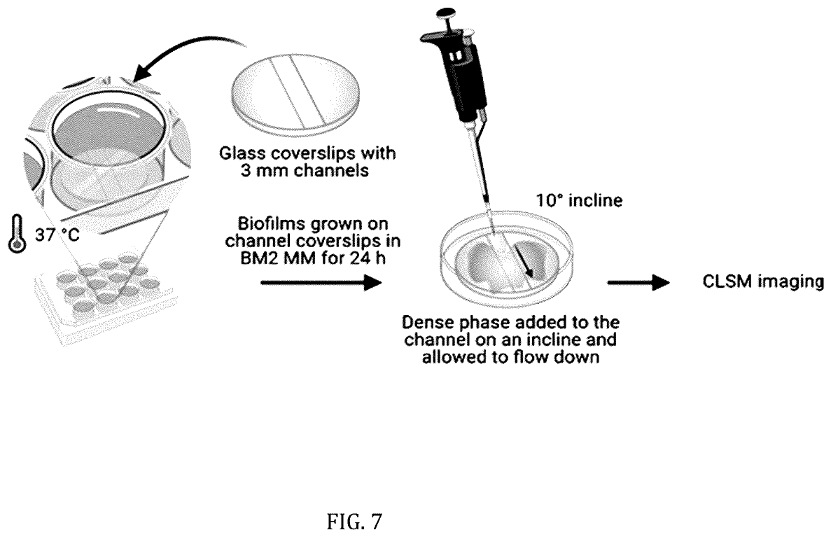

Biofilms from P. aeruginosa were grown on channels formed within glass coverslips as shown in FIG. 7. The bacteria were stained with cell-permeable dye DMAO to visualize the bacteria, and using a micropipette, a 20 μL drop of coacervate was added to one end of the channel underwater, as shown in FIG. 7. When this system was given a small incline (10°) surprisingly, it was observed that the dense phase ‘rolled’ off the surface of the biofilm, through the length of the channel instead of spreading on the biofilm. This was unexpected since it has been demonstrated previously that the dense phase is a low interfacial energy fluid that readily spreads underwater glass. Without wishing to be bound by theory, it is likely that the conditioning film of bacterial adhesions and secreted polysaccharides from the biofilm lowers the surface energy of the glass or forms a hydration layer that may prevent the dense phase from spreading on the surface.

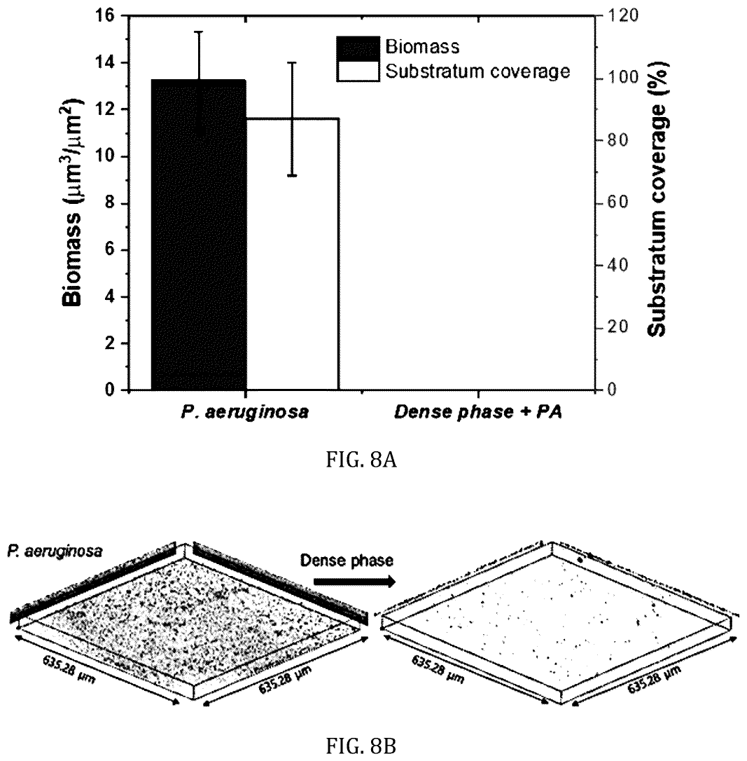

The COMSTAT plugin in ImageJ was used to quantify and compare biofilm biomass, which represents the area-normalized total volume of biofilm in the sample, and substratum coverage, which quantifies the area occupied by the biofilm on the layer atop the substrate relative to total area. As shown in FIG. 8A, the biofilm biomass in the channels decreased by 99.9% from 13.2±2 μm to 0.004±0.003 μm, while the area occupied by the biofilm (substratum coverage) decreased by 99.9% from 87±18% to 0.09±0.07%. FIG. 8B shows 3D renders of confocal stacks before (left) and after (right) the addition of coacervate dense phase on biofilms of P. aeruginosa grown under static conditions on channel coverslips.

### Example 2—K. pneumoniae, E. coli, and S. aureus Studies

Biofilms of gram-negative bacteria Klebsiella pneumoniae and Escherichia coli, and gram-positive bacteria Staphylococcus aureus were grown in accordance with the with the setup described in Example 1. Each of the biofilms from each bacterium were subjected to the dense phase of the polymer coacervate.

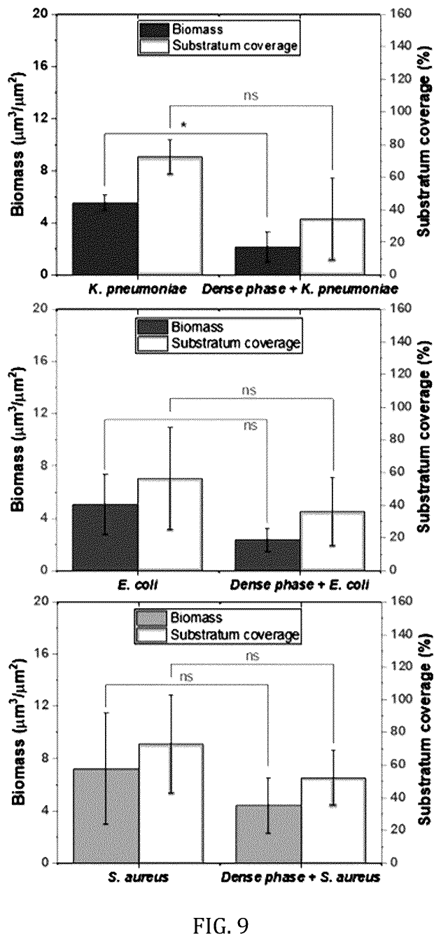

For K. pneumoniae, the biofilm-biomass decreased by 70±25%, while the differences in the substratum coverage before and after the application of the dense phase were not statistically significant, as shown in FIG. 9.

For E. Coli and S. aureus, no statistically significant differences before and after the addition of the dense phase to each of the biofilms was observed, as shown in FIG. 9.

Example 2 demonstrates that for each bacterial species, the structure, chemical composition, and biomechanical characteristics of individual biofilms vary greatly because different bacteria use different biochemical pathways to synthesize and construct their surface adhesions and biofilm matrix components. K. pneumoniae forms polysaccharide-rich biofilms. E. coli and S. aureus form biofilms with protein-rich matrices. Aside from the differences in the chemical compositions of the biofilm matrices, which could play a part in mediating interactions with the dense phase, the differences in the mechanical properties of the biofilms and their substrate adhesion could also be responsible for differences in the amount of their displacement by dense phase.

### Example 3—Shear and Interfacial Studies

The application of mechanical shear is known to cause significant loss of biomass in mature biofilms. It is also known that biofilms grown under fluid flow and high shear stresses are found to be thinner, but denser and more resistant to external mechanical perturbations.

If the interfacial properties of the dense phase do play a role in its ability to remove P. aeruginosa biofilms from a surface, then, changing the surface chemistry (and in turn the surface energy) of a substrate should also lead to changes in the biofilm displacement. Glass substrates were chemically functionalized by growing self-assembled monolayers (SAMs), as shown in FIG. 4. PEG-SAMs and PETS-SAMs were chosen as representative ‘hydrophilic’ (water contact angle=45.7°±1.3° and ‘hydrophobic’ (water contact angle=80.4°±3.7°) surfaces. The dilute phase, which contains approximately 2% w/w polymer had a contact angle of 24°±3.3° on glass, 26.2°±1.6° on PEG-SAMs, and 55°±2.7° on PETS-SAMs in air. The decrease in dilute phase contact angle relative to water on PEG-SAMs and PETS-SAMs may be attributed to the amphiphilic composition of the polymer. The dense phase, likely due to its higher concentration of polymer (˜54% w/w) and higher viscosity had a contact angle of 34.5°±0.8° on glass, 57.7°±3.1° on PEG-SAMs and 63.56°±2°.

Shear and interfacial studies used a 3-channel laminar flow setup as shown in FIG. 4. The setup also enabled longer periods for growth of biofilms, on different substrates, and with continuous nutrient supply, which enabled their direct visualization before and after treatments with minimal damage to biofilm structure during sample preparation. In general, after biofilm culture, 50 μL of dense phase was injected into the inlet, inclined at 10° and allowed to roll down the length of the channel (40 mm long, 4 mm wide, 1.6 cm2 growth area), after which the channels were rinsed with 1 mL water (flowing at 1 mL/min) and the biofilms were imaged under a microscope.

To test if the biofilm removal by the dense phase was due to the viscosity (induced shear stress) of the dense phase, the tests also employed 20% w/v aqueous PVA solution as a hydrophilic viscosity control and SYLGARD™ 184 Part A as a hydrophobic viscosity control (η₀˜5.1 Pa-s). For the duration of the experiments (˜10 min), both 20% PVA and SYLGARD™ remained immiscible underwater, were more viscous than the dense phase, and had a greater density than water. Both 20% PVA and SYLGARD™ did not flow in the channels under the influence of gravity (at 10° incline) and were removed during the washing step. This is why regions of the channels where 20% PVA or SYLGARD™ was physically present were imaged.

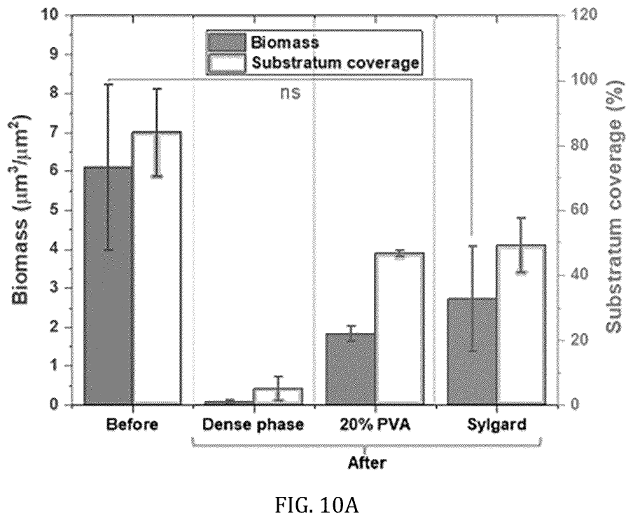

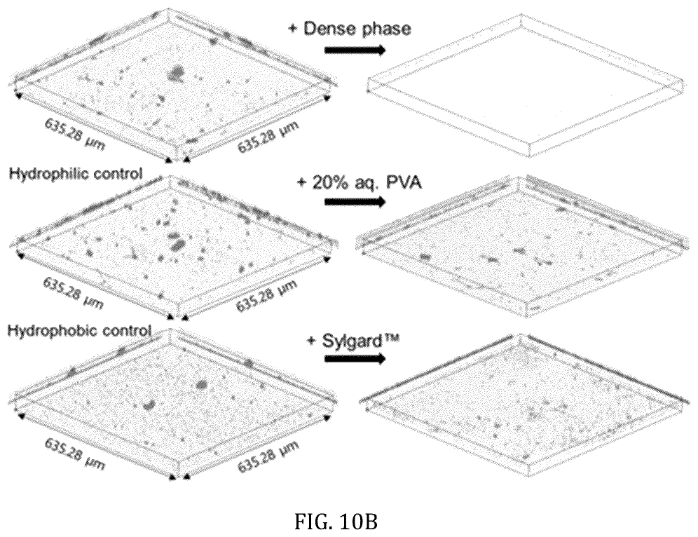

As seen in FIG. 10A, for biofilms grown under hydrodynamic flow conditions, the dense phase was still able to displace 98.5±0.8% biomass from the surface leading to 94±4% decrease in substratum coverage. While 20% PVA led to 70±3% decrease in total biomass and 56±1% surface clearance. The difference in biofilm biomass before and after the addition of SYLGARD™ 184 Part A was not found to be statistically significant, but the substratum coverage was reduced by 41±10%. FIG. 10B shows 3D render of confocal z-stacks of P. aeruginosa biofilms before and after addition of the dense phase, 20% PVA, and SYLGARD™ 184 Part A. These observations provide an understanding that while the flow of a viscous material through the biofilm could lead to a significant loss of biomass, viscosity alone is insufficient to explain the biofilm clearance by the dense phase. This indicates a role played by the interfacial properties of the dense phase in displacing biomass from the surface.

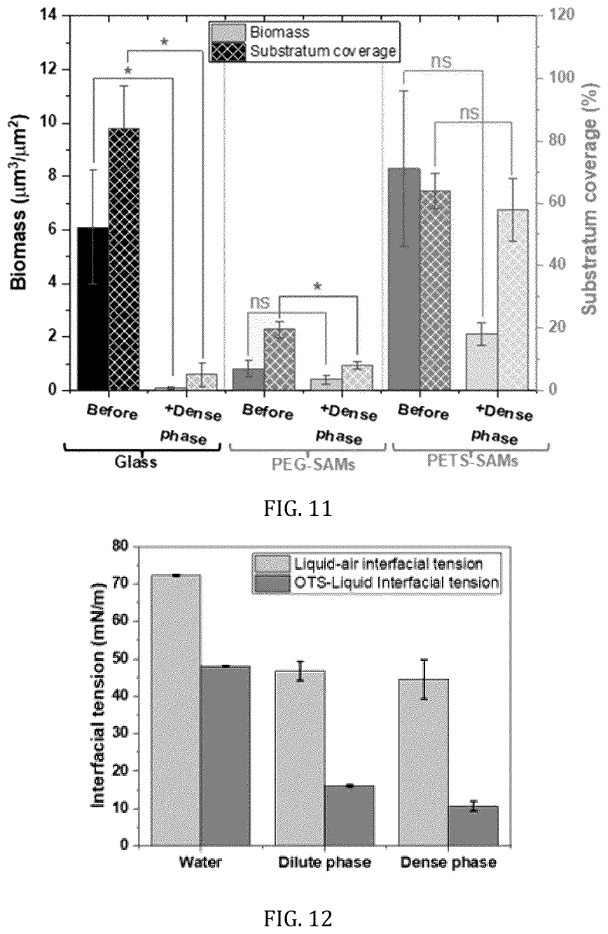

P. aeruginosa biofilms were cultured on PEG-SAMs and PETS-SAMs functionalized glass under hydrodynamic media flow and subjected them to similar treatment with the dense phase as described in the section above. As shown in FIG. 11, the biofilm biomass and surface coverage are vastly different for glass, PEG-SAMs, and PETS-SAMs. The biomass and the substratum coverage are the least for biofilms grown on PEG-SAMs. Since PEG-functionalized surfaces are commonly employed for antifouling and microbial-repellent applications due to the presence of a hydration layer this result is unsurprising. Within the 72 h of culture, bacteria do end up overcoming this hydration barrier to eventually attach and initiate biofilm formation even on PEG-SAMs, which goes to say that PEG-based antifouling strategies may not provide long-term microbial repellency. For biofilms grown on PEG-SAMs, the dense phase does not cause significant biomass reduction but does lead to almost 60±6% decrease in biofilm substratum coverage.

Previously, it was reported that biofilms are generally denser and more robust on hydrophobic surfaces relative to hydrophilic surfaces. In the course of these experiments, no significant differences between the biomass or surface coverage of biofilms grown on glass or PETS-SAMs was observed. Further, no statistically significant differences between the biofilms (on PET-SAMs) before and after the application of the dense phase were observed. With these observations, the amount of biofilm displaced by the dense phase underwater, appears to decrease with decreasing surface energy (increasing surface hydrophobicity). As such, both viscosity and interfacial properties of the dense phase play a role in its ability to displace biofilms attached to a surface.

### Example 4—Interfacial Interaction Study

Interfacial interactions may be measured using the custom-built JKR (Johnson, Kendall, and Roberts contact model) setup for calculating the work of adhesion to fracture the biofilm-substrate interface, as described above, and shown in FIG. 6. The setup contains a hard glass hemisphere attached to a load cell, which is brought in contact with the biofilm grown on a soft PDMS substrate (G=0.7 MPa) under a medium of interest.

Biofilms are very soft and thin materials when grown on a hard surface like glass, thus the measured pull-off forces were almost negligible due to minimal contact area in hard-hard contact. Therefore, biofilms were cultured on soft and hydrophobic PDMS surface for these experiments. Additionally, by using SAMs to functionalize the glass lens (and thereby alter its surface energy), Applicant was able to probe the work of adhesion to fracture biofilm-substrate interface as a function of substrate surface energy (which would be inversely proportional to the water contact angle). To minimize the effects of viscosity of the dense phase, the pull-off measurements under the dilute phase, with the assumption that the effect of interfacial properties of the dilute phase on pull-off forces should not be dissimilar to that of the dense phase.

On a representative n-octadecyltrichlorosilane (OTS) functionalized surface, the interfacial tensions of both the dilute phase and dense phase are comparable (γ_{Dilute phase-OTS}=16±0.3 mN/m and γ_{Dense phase-OTS}=10.6±1.3 mN/m while γ_{Dilute phase-air}=46.7±2.6 mN/m and γ_{Dense phase-air}=44.5±5.3 mN/m). Additionally, the dilute phase can also displace biofilms from surfaces, alike the dense phase.

In these experiments, the flow rate of the dilute phase in the channels was selected to mimic the shear-forces on the biofilm due to the flow of the viscous dense phase. Thus, a flow rate of 20 μL/min was chosen since it approximated the rate of flow of dense phase droplet through the length of the channel and the subsequent flow rates (200 μL/min and 2000 μL/min) were chosen to amplify the shear forces on the biofilm.

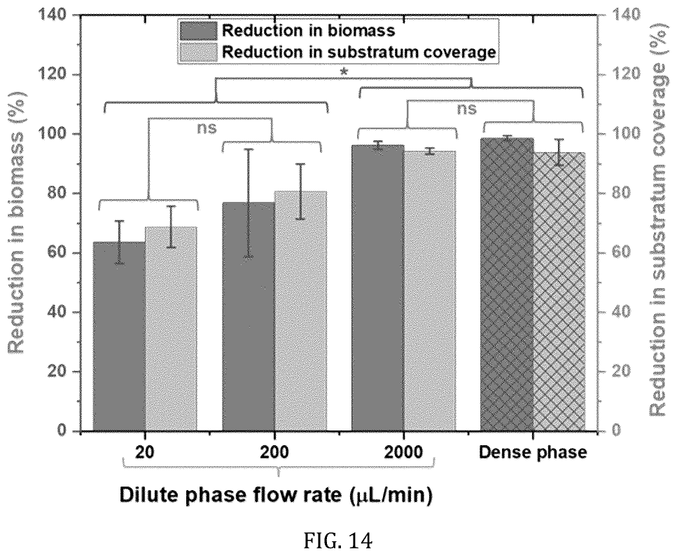

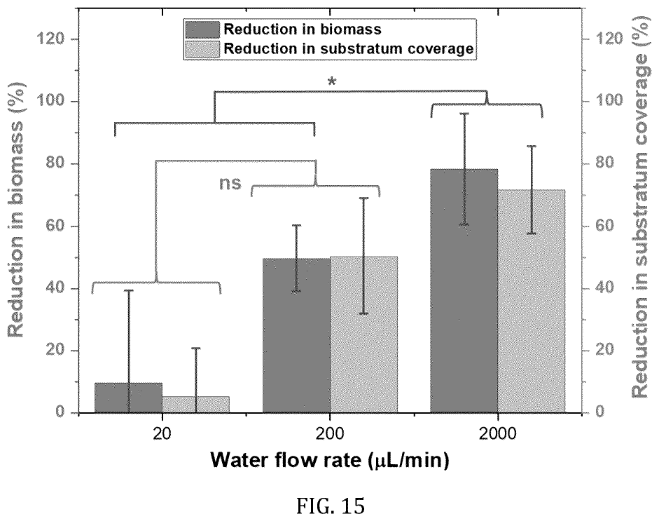

As shown in FIG. 14, the dilute phase flowing at 20 μL/min led to 63±7% reduction in biofilm biomass and 68±7% reduction in substratum coverage of the biofilm, while at 200 μL/min, we did not see statistically significant differences in biomass or substratum coverage compared to 20 μL/min. Increasing the flow rate to 2000 μL/min lead to an almost 96±1% decrease in biomass and 94±1% decrease in surface coverage, which is similar to the effect of the dense phase. Water was used as a control for this experiment. As shown in FIG. 15, flowing water through the channels at 20 μL/min has insignificant effect on the biofilm and led to 9±29% reduction in biomass and 5±15% reduction in surface coverage. Increasing the flow rate to 200 μL/min did not lead to statistically significant results. At high flow rates of 2000 μL/min, a 78±18% reduction in biomass and 71±14% reduction in biofilm surface coverage due to water was observed. These results indicate that while both water and dilute phase flow can lead to shear forces and subsequent loss of biomass in biofilms, the presence of polymer in the dilute phase cause greater displacement of biofilm from the surface than with water alone.

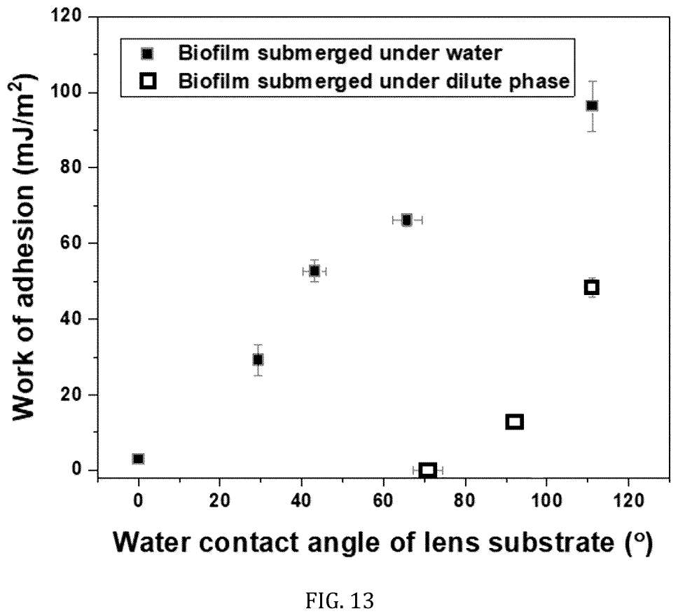

The dilute phase and dense phase have similar interfacial properties and that the dilute phase can functionally mimic the dense phase at extremely high flow rates. This property was used to investigate the effect of the dilute phase on the work of adhesion to fracture the biofilm-substrate interface. As shown in FIG. 13, the work of adhesion trends almost linearly with the water contact angle of the substrate, implying that the energy required to fracture the biofilm/hydrophobic surface (OTS) interface is much higher than that required in the case of a hydrophilic surface (glass). These results are consistent with prior reports that biofilms stick better to hydrophobic surfaces. In presence of the dilute phase, the work of adhesion to fracture the biofilm-substrate interface decreases dramatically-indicating that the constituent polyester of the dilute and dense phase is surface active and may interfere with the adhesive bonds that the biofilm makes with the substrate. In fact, the experiment was not able to detect significant pull-off forces for substrates with water contact angles below 70° in presence of the dilute phase. As seen underwater, the work of adhesion to fracture biofilm from the substrate increases with substrate hydrophobicity, which implies that it gets harder for the dilute phase to remove biofilms growing on hydrophobic surfaces than hydrophilic surfaces. As such Example 4 demonstrates that the dilute phase, and by extension, the dense phase reduces the energy (work of adhesion) required to fracture the biofilm-substratum interface. Since the work of adhesion is significantly higher for hydrophobic surfaces relative to hydrophilic surfaces, less biofilm removal on hydrophobic surfaces than on hydrophilic surfaces was observed. Biofilm removal by the dense phase is thus mediated via both interfacial interactions and flow-induced shear forces on the biofilm.

1. A method for disrupting and removing a biofilm from a contaminated substrate, the method comprising:
contacting the biofilm and the contaminated substrate with a polyester coacervate composition.

2. The method of claim 1, wherein the polyester coacervate composition comprises:
an aqueous solution, and
a polyester coacervate dispersed within the aqueous solution.

3. The method of claim 1, wherein the biofilm and the contaminated substrate are exposed to water.

4. The method of claim 2, wherein the polyester coacervate composition includes an additive, wherein the additive is an anti-microbial agent.

5. The method of claim 4, wherein the anti-microbial agent is an antibiotic encapsulated within the polyester coacervate.

6. The method of claim 1, wherein 55 percent or greater of a biomass of the biofilm is removed from the contaminated substrate after being contacted with the polyester coacervate solution.

7. The method of claim 1, wherein from 55 percent to 99.9 percent of a biomass of the biofilm is removed from the contaminated substrate after being contacted.

8. The method of claim 1, wherein an area of the contaminated substrate including the biofilm is decreased by from 55 percent to 99.9 percent after being contacted with the polyester coacervate solution.

9. The method of claim 1, wherein the step of contacting includes coating, spreading, injecting, depositing, flowing, or otherwise bringing the polyester coating coacervate composition into contact with the biofilm.

10. The method of claim 1, wherein the biofilm is formed from one of K. pneumoniae, E. coli, S. aureus, and Pseudomonas including P. aeruginosa, P. fluorescens.

11. A composition comprising:
an aqueous solution,
a polyester coacervate dispersed within the aqueous solution, and
an additive.

12. The composition of claim 11, wherein the polyester coacervate is represented by Formula I: 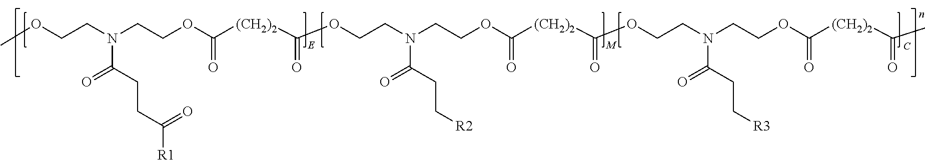
Formula I
where R1 is selected from moieties that include an N- or N,N-substituted secondary or tertiary amide, R2 and R3 are independently selected from aliphatic groups, unsubstituted aromatic functional groups, and aromatic groups functionalized with one or more hydroxyl groups, n is from about 10 to about 500, E is from about 60 to about 95, M is from 0 to about 40, C is from about 5 to about 40, and where the sum of M+E+C is 100.

13. The composition of claim 11, wherein the polyester coacervate includes a unit represented by, one of:

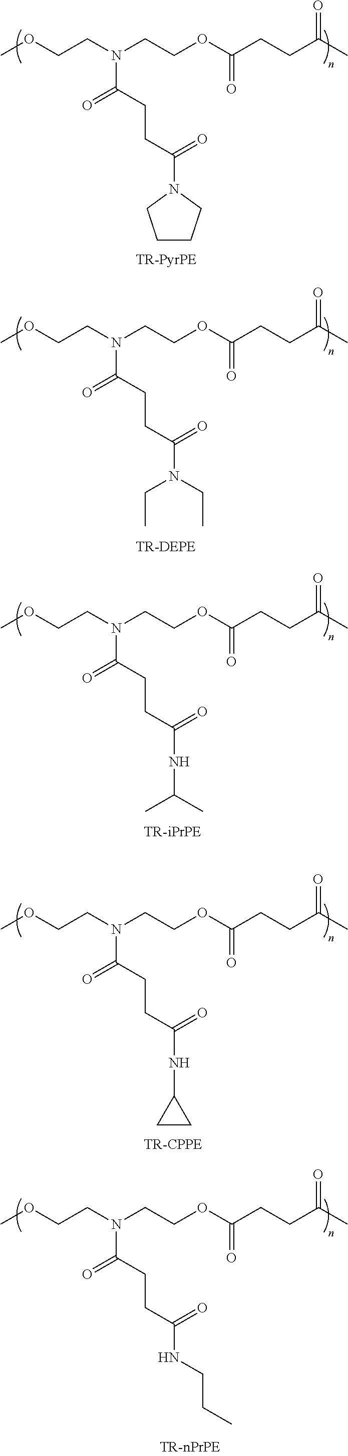

14. The composition of claim 11, wherein the additive is encapsulated by the polyester coacervate.

15. The composition of claim 11, wherein the additive is an anti-microbial agent.

16. The composition of claim 11, wherein the additive is ciprofloxacin.

17. The composition of claim 11, wherein the polyester coacervate has a density greater than water.
